(19) United States
(12) Patent Application Publication
(10) Pub. No.: US 2011/0126839 A1
(43) Pub. Date: Jun. 2, 2011

(54) RESPIRATORY FACEMASK WITH SLIDING ENDOTRACHEAL TUBE HOLDER

(76) Inventor: Walter Levine, Delray Beach, FL (US)

(21) Appl. No.: 12/956,627

(22) Filed: Nov. 30, 2010

Related U.S. Application Data
(60) Provisional application No. 61/265,138, filed on Nov. 30, 2009.

(51) Int. Cl. A61M 16/04

(52) U.S. Cl. 128/207.14

(57) ABSTRACT

A respiratory therapy facemask configured to be positioned against a patient's face and adjacent the patient's mouth, includes a face plate configured for attachment to the patient and defining an opening, the face plate including a laterally extending track defining a slot. A tube rest assembly is configured for being slidably engaged in the slot and for securely accommodating an endotracheal tube. The tube rest assembly is configured for directly engaging the track for lateral movement in the opening.

### RELATED APPLICATION

This application claims priority under 35 USC 119(e) based on prior U.S. Ser. No. 61/265,138 filed Nov. 30, 2009.

### BACKGROUND

The present invention relates generally to apparatus for facilitating the treatment of patients with respiratory ailments or who need breathing assistance in the course of other medical treatment, and more specifically to an improved respiratory facemask for more securely retaining an endotracheal or laryngeal tube (hereinafter “endotracheal tube”) in the patient's mouth.

In conventional respiratory therapy applications, tape is often used to secure the endotracheal tube in place. The use of tape in such applications has several drawbacks. For example, tape loses adhesion when patients perspire and/or have facial hair growth, or on patients with facial burns. In addition, tape is not recommended for use on elderly patients since the tape could remove the facial skin upon tape removal.

To reduce the use of tape in such applications, facemasks have been developed which are strapped to the patient's head and provide a relatively stable platform for mounting the endotracheal tube in place. Exemplary prior art facemasks are disclosed in facemask U.S. Pat. Nos. 5,345,931 and 4,744,358, which are incorporated by reference. One drawback of the prior art products is that the endotracheal tube still became loose over time, as medical technicians perform routine monitoring and sanitation tasks in administering to the patient. As such, medical technicians still resort to the use of tape to better secure the treatment tube in place, even when a facemask is used. Another drawback is the relatively high manufacturing cost of the prior art products.

### SUMMARY

The present respiratory facemask addresses drawbacks of prior art products, more securely fixes the endotracheal tube to the patient, and preferably eliminates need for tape to secure the tube in place. Structural features are provided for securing the endotracheal tube against unwanted horizontal and vertical movement relative to the patient's face. In addition, the nasogastric tube is optionally secured by the present facemask. With the present side-to-side tube mount motion, oral hygiene can now be performed without the need to disassemble the device from the patient's face. Despite such lateral movement of the tube mount, the present facemask prevents unwanted vertical tube movement relative to the patient's face, and maintains fixation of the endotracheal cuff location, thus preventing excessive oral secretion transfer during the period that the tube is unsecured. The transfer of excessive oral secretions below the endotracheal cuff is currently a location for the transfer of contamination. Also, tracheal and laryngeal trauma is reduced.

With the present facemask, pressure upon the patient's face is more evenly distributed, lessening the possibility of pressure necrosis. Instead of hard plastic, nonporous foam rests against the patient's skin, allowing frequent facial hygiene, thus preventing bacterial growth. Another feature of the present facemask is the soft, rubber-like bite block that guards against biting, prevents tube occlusion, and inhibits patients from cutting off airflow without causing discomfort. The present facemask includes a bite block lip protector for enhanced patient comfort.

More specifically, a respiratory therapy facemask configured to be positioned against a patient's face and adjacent the patient's mouth includes a face plate configured for attachment to the patient and defining an opening, the face plate includes a laterally extending track defining a slot. A tube rest assembly is configured for being slidably engaged in the slot and for securely accommodating an endotracheal tube. The tube rest assembly is configured for directly engaging the track for lateral movement in the opening.

In another embodiment, a respiratory therapy facemask configured to be positioned against a patient's face and adjacent the patient's mouth includes a face plate configured for attachment to the patient and defining an opening, the face plate has a laterally extending track defining a slot. A tube rest assembly is configured for being slidably engaged in the slot and for securely accommodating an endotracheal tube. At least one clip is provided for engaging the tube rest assembly from a rear side of the face plate for vertically stabilizing the tube rest as it laterally slides relative to the face plate.

In yet another embodiment, a respiratory therapy facemask configured to be positioned against a patient's face and adjacent the patient's mouth includes a face plate configured for attachment to the patient and defining an opening, the face plate includes a laterally extending track defining a slot. A tube rest assembly is configured for being slidably engaged in the slot and for securely accommodating an endotracheal tube. At least one of the tube rest assembly and the face plate having ribs for facilitating the sliding engagement in the slot. A slide clip retains the tube rest assembly to the face plate and slides with the tube rest assembly in the slot, with the face plate sandwiched between the clip and the tube rest assembly. A hook clip engages the tube rest assembly and has a hook clip slidably engaging a lower edge of the opening and preventing unwanted vertical and forward movement of the tube rest assembly relative to the face plate.

### BRIEF DESCRIPTION OF THE DRAWINGS

FIG. 1 is a lower front perspective view of the present respiratory facemask;

FIG. 2 is an upper rear perspective view of the facemask of FIG. 1 without the pads;

FIG. 3 is a front elevation of the present facemask;

FIG. 4 is a top view of same;

FIG. 5 is a left side elevation of same;

FIG. 6 is a rear elevation of the present facemask;

FIG. 7 is a partial cross-section taken along the line 7-7 of FIG. 6 and in the direction indicated generally;

FIG. 8 is a fragmentary enlarged cross-section of FIG. 7;

FIG. 9 is a right side elevation of the present facemask tube rest assembly;

FIG. 10 is a rear elevation of the tube rest of FIG. 9;

FIG. 11 is a cross-section taken along the line 11-11 of FIG. 9 and in the direction indicated generally;

FIG. 12 is a section taken along the line 12-12 of FIG. 10 and in the direction indicated generally;

FIG. 13 is a fragmentary front elevation of the present tube rest assembly;

FIG. 14 is an exploded top perspective view of the present tube rest and pad;

FIG. 14A is an assembled top perspective view of the present tube rest and pad;

FIG. 15 is a front bottom perspective view of the present hook clip;

FIG. 15A is a rear bottom perspective view of the present hook clip of FIG. 15;

FIG. 16 is a rear perspective view of the present spring clip;

FIG. 16A is a front perspective view of the spring clip of FIG. 16

FIG. 17 is a front elevation of the present facemask face plate;

FIG. 18 is a right side view of the face plate shown in FIG. 17;

FIG. 19 is a cross-section taken along the line 19-19 in FIG. 17 in the direction indicated generally;

FIG. 20 is a fragmentary top rear perspective view of the present tube rest assembly;

FIG. 21 is a rear elevation of the tube rest assembly shown in FIG. 20;

FIG. 22 is a fragmentary overhead plan view of the tube rest assembly shown in FIG. 20;

FIG. 23 is a top perspective view of the present strap and bite block; and

FIG. 24 is a right side elevation of the strap and slide block shown in FIG. 23.

### DETAILED DESCRIPTION

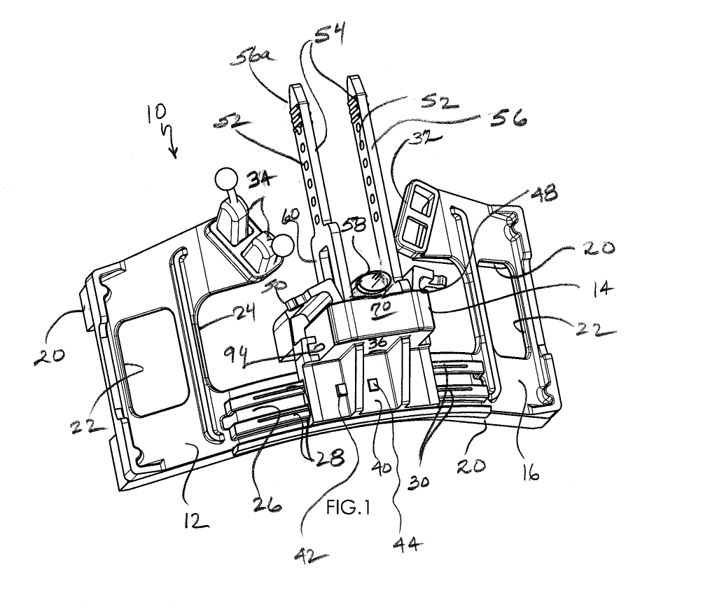

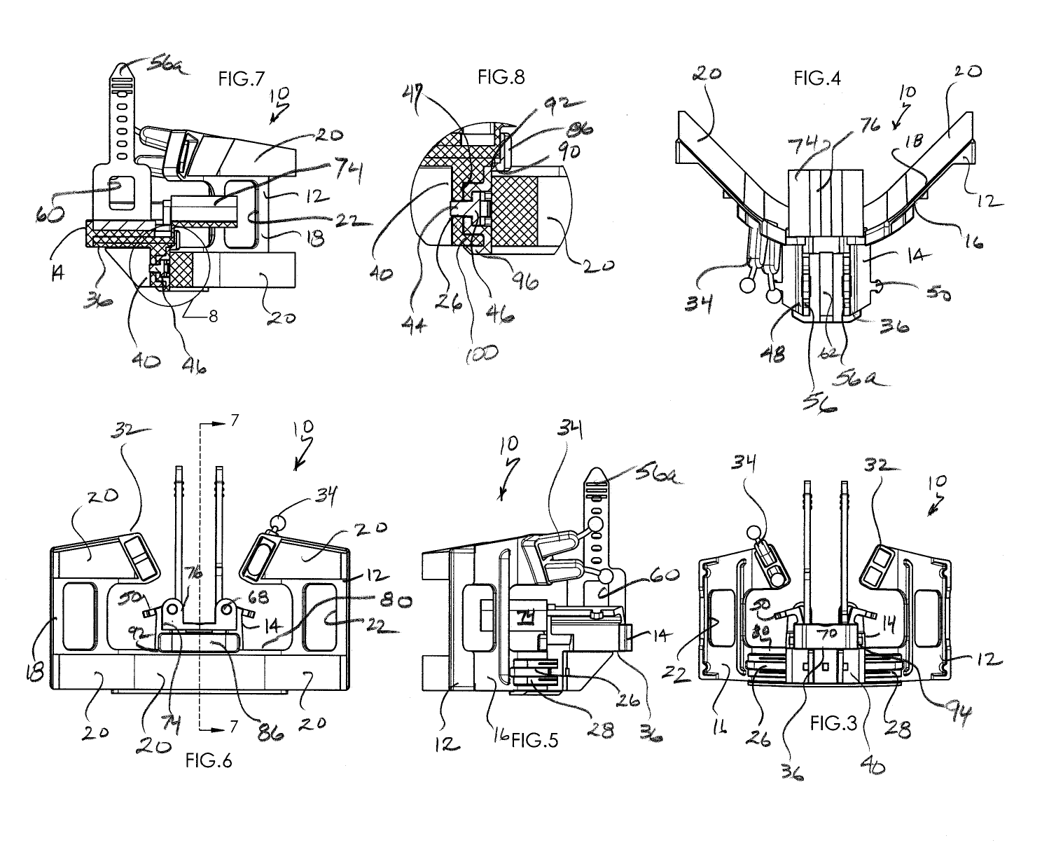

Referring now to FIGS. 1-7, the present facemask, generally designated 10, includes a face plate 12 configured for being secured to the face of a patient by a harness (not shown) as is well known in the art and described in U.S. Pat. Nos. 5,345,931 and 4,744,358 incorporated by reference. The main purpose of the present facemask 10 is to secure an endotracheal or laryngeal tube (hereinafter “endotracheal tube”) in place in the patient's mouth, the tube being separate from the facemask 10. A main feature of the present facemask 10 is that the endotracheal tube is actually secured to a tube rest assembly 14, which directly contacts and is laterally slidable relative to the face plate 12 to permit the performance of oral hygiene operations on the patient while the tube is maintained in the patient's trachea and mouth for therapeutic concerns. It is contemplated that both the face plate 12 and the tube rest assembly 14 are made of generally rigid, self-supporting plastic materials as are known in the art, including, but not limited to polyvinylchloride (PVC).

The face plate 12 includes a front surface 16 and a rear surface 18, the latter designed to contact the patient's face and as such is provided with at least one pad 20 made of relatively soft material such as polyurethane foam or the like to facilitate patient comfort. In the preferred embodiment, the patient's cheeks and chin surface are contacted by the rear surface 18, which is covered by the pads 20, numbering three. However, the number, placement and composition of the pads 20 may vary to suit the application. Also, the face plate 12 is provided with at least one and preferably a pair of openings 22 for attaching the harness (not shown) used to secure the facemask 10 to the patient's head, as is known in the art.

Between the openings 22 is a preferably central opening 24 which defines a space for lateral movement of the tube rest assembly 14. Below the central opening 24 is a generally horizontally or laterally extending slot 26 defined on upper and lower ends by a track 28, preferably integrally joined to the face plate 12. Referring now to the front surface 16 of the face plate 12, the track 28 is provided with outwardly projecting ribs 30 which facilitate lateral sliding of the tube rest assembly 14 as will be described below. Above the track 28, an upper border of the central opening 24 is provided with a pair of clip mounts 32 configured for accommodating clips 34 in a friction-fit engagement for securing a naso-gastral intestinal (NGI) tube (not shown). The NGI tube, which is distinct from the main, endotracheal tube, is basically jammed into a wedge-like foam-lined portion of the clip 34 and held by a friction fit.

Referring now to FIGS. 1, 2, 7, 8, 16 and 16A, the tube rest assembly 14 includes a forwardly located tube rest assembly body 36 and a relatively rearwardly located bite block 38 connected to the tube rest assembly body, preferably by having a portion being integrally molded, however assembly of various components by chemical adhesive, ultrasonic molding or fasteners is also contemplated. In the context of the present facemask 10, “forwardly” refers to away from a patient's face, and “rearwardly” refers to toward the patient's face.

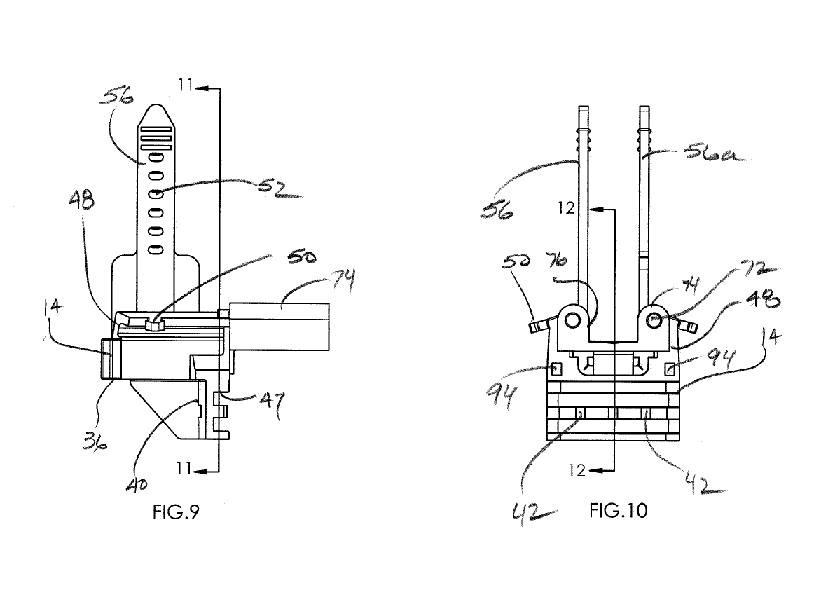

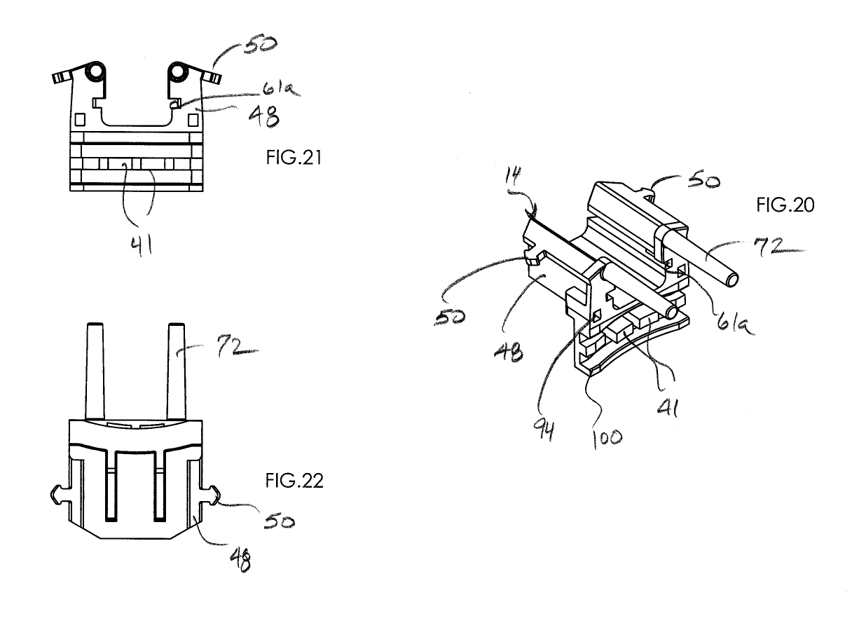

An important feature of the present facemask 10 is that the tube rest assembly 14 is slidably secured to the face plate 12 at several locations along or adjacent a vertical axis defined by points of engagement of the two components. When viewed from the front surface 16, the tube rest assembly body 36 preferably includes a front panel 40 having at least one opening 42 for accommodating optionally barbed prongs 44 secured to a snap clip 46 slidingly engaged on the rear surface 18 on the track 28. The number and placement of the prongs 44 may vary to suit the situation, and in one embodiment, there are preferably three prongs 44 received in corresponding openings 42 in the front panel 40. The engagement of the prongs 44 in the openings 42 secures the tube rest assembly 14 to the face plate 12 for lateral sliding motion facilitated by the engagement of an inner surface 47 (FIG. 9) of the front panel 40 with the ribs 30. It is contemplated that the prongs 44 have hook or barb-shaped ends (FIG. 16) for enhancing the engagement between the snap clip 46 and the tube rest assembly 14, preventing unwanted vertical movement of the tube rest assembly relative to the face plate 12. In addition, the front panel has at least one rearwardly projecting tab 41 (FIG. 20) that matingly engages holes 43 (FIG. 16) in the snap clip 46.

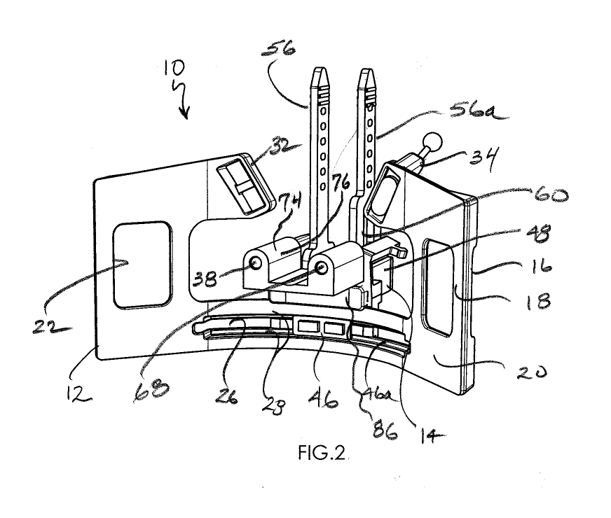

As also seen in FIG. 16, the clip 46 also has elongate ribs 46a for facilitating this lateral sliding action. As seen in FIGS. 2 and 6, the clip 46 slides behind the pad 20 when viewed from the rear surface 18, or forward of the patient, so that the patient's skin is not exposed to the sliding movement. It is contemplated that the ribs 46a may alternatively be placed on the rear surface 18 of the face plate 12 near the track 28, and adjacent the slot 26 (FIG. 2).

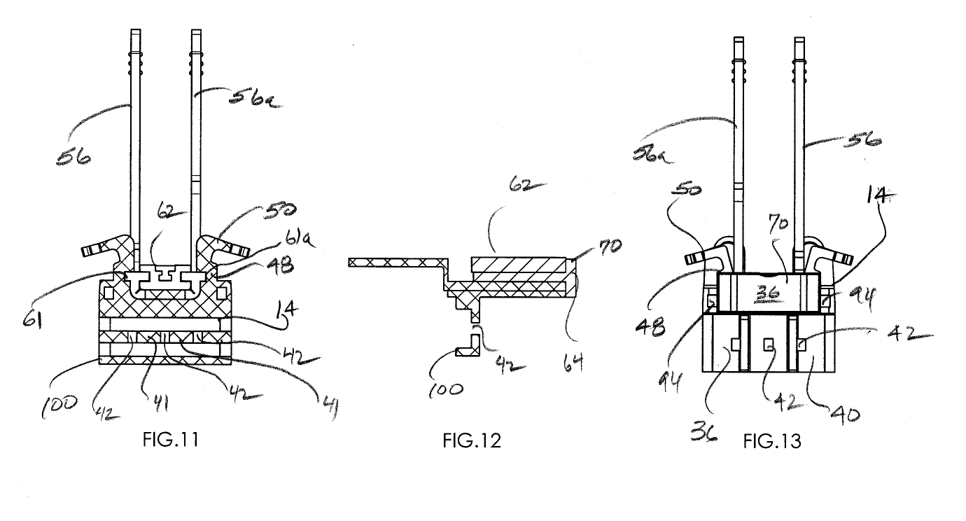

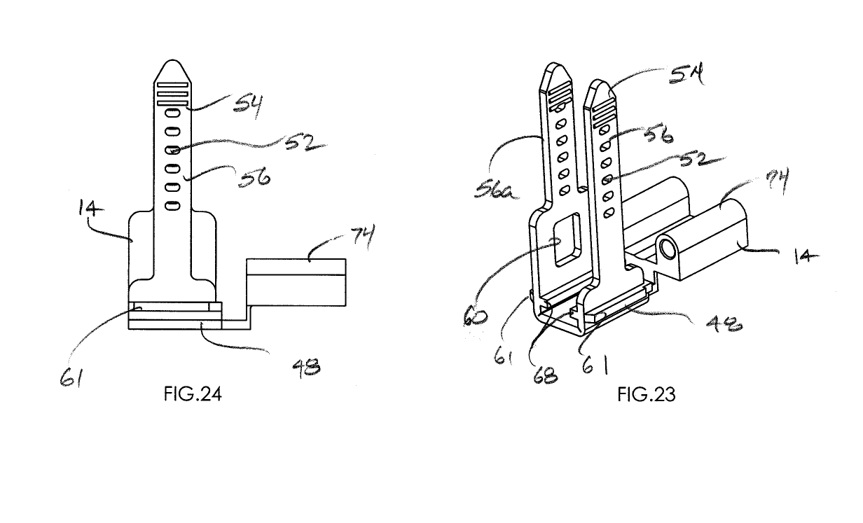

Secured to an upper margin of the front panel 40 is a forwardly projecting tube holder 48 having a generally “U”-shape when viewed from the front. At least one leg of the U includes an angled, arrow-shaped strap anchor 50. Each anchor 50 is constructed and arranged to engage a desired one of a series of linearly spaced locking apertures 52 on each end 54 of an endotracheal locking strap 56 (FIGS. 23 and 24). The two ends 54 overlap each other to secure an endotracheal tube 58 (FIG. 1) in place. One strap 56a includes an opening 60 for receiving the other strap 56 in this overlapping manner. As seen in FIGS. 11, 13 and 23, the straps 56, 56a are preferably fastened to the tube holder 48 by engagement between laterally extending ribs 61 with corresponding grooves 61a on the tube holder 48 (FIGS. 20 and 21).

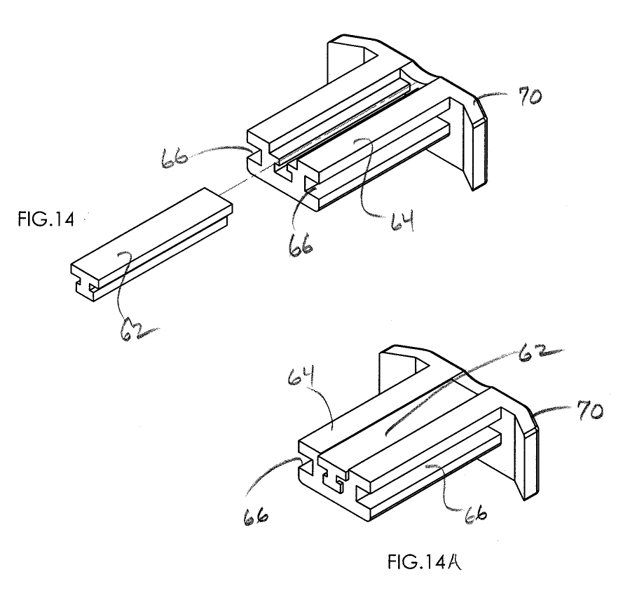

Referring now to FIGS. 11, 12, 14 and 14A, the endotracheal tube 58 rests upon a relatively soft tube rest insert 62 made of rubber-like or low Durometer resilient polyvinyl chloride (PVC) material. Since the endotracheal tube 58 is also typically made of PVC, the inherent self-adhesive nature of PVC enhances the adhesion of the tube to the tube rest insert, and more securely retains the tube in position. The tube rest insert 62 has a generally uneven “I-beam” construction when viewed in cross-section and is held in place on a relatively rigid or higher Durometer tube rest 64 by a tongue-in-groove relationship best seen in FIG. 14. Once slidably installed in the relatively hard plastic tube rest 64, the tube rest insert 62 is generally flush with the tube rest 64. The tube rest 64 is in turn slidably engaged into the tube holder 48 by a tongue-in-groove arrangement defined by lateral grooves 66 in the pad which slidingly engage horizontal ribs 68 in the tube holder 48 (FIG. 23). As seen in FIGS. 13 and 14, the tube rest 64 has a front panel 70. Thus, when the endotracheal tube 58 is placed upon the tube rest insert 62, the tube is secured in part by this self-adhesion and in part by the endotracheal locking straps 56.

Referring to FIGS. 2, 6 and 20-24, rearwardly projecting from the tube holder 48 are a pair of parallel, spaced rods 72 upon which are matingly engaged a bite block pad 74. The bite block pad 74 is also generally “U”-shaped when viewed from the rear (FIG. 10) and is made of a generally soft, rubber-like, PVC plastic material to be comfortably inserted into the patient's mouth during respiratory therapy, which refers to any type of medical treatment, including but not limited to emergency, first responder, paramedic use, and anesthesia. The pad 74 is friction fit upon the rods 72, and may be secured if desired using chemical adhesive or the like. In addition, the pad 74 defines a rest 76 for receiving the endotracheal tube 58.

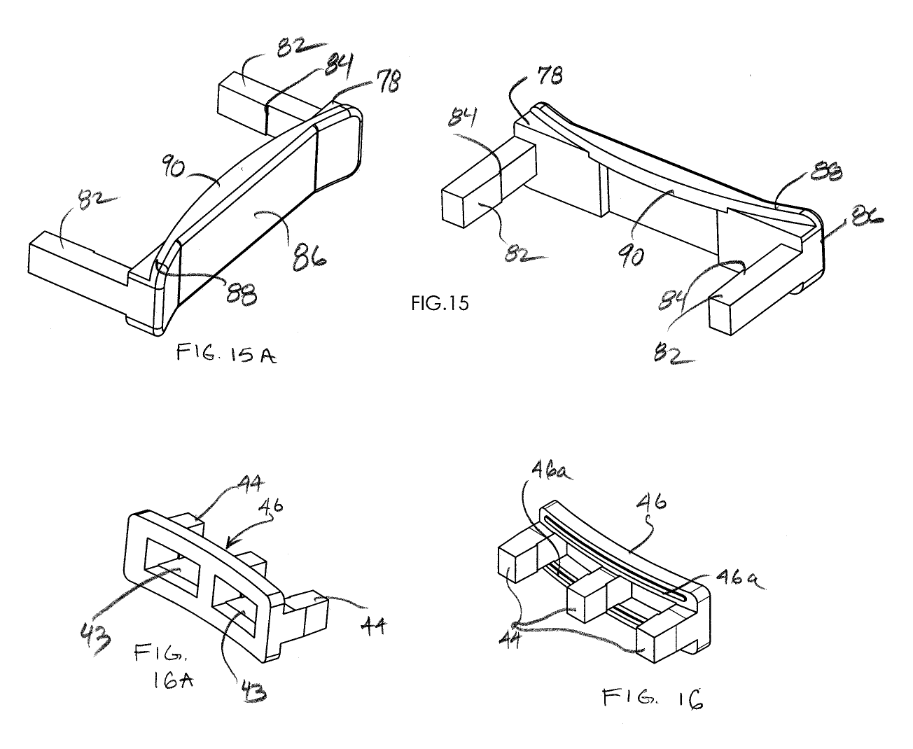

Referring now to FIGS. 2, 7, 8, 15 and 15A, the tube rest assembly 14 is also more securely and slidably held to the face plate 12 by at least one hook clip 78. The hook clip 78 is disposed near a lower edge 80 of the central opening 24 and closer to the tube holder than the track 28. At least one and preferably a pair of lugs 82, optionally provided with barbs 84 are connected to a main panel 86. A lower edge 88 of the main panel is provided with a hook rib 90 that slidably engages an undercut 92 on the rear surface. The lugs 82 matingly engage openings 94 in the tube rest assembly 14 (FIGS. 11, 13 and 20).

The use of the hook clip 78 supplements the snap clip 46 and helps prevent unwanted forward or vertical movement of the tube rest assembly 14 relative to the face plate 12 during respiratory therapy, or during oral hygiene conducted by a medical technician, at which time the tube rest assembly 14 is laterally moved along the track 28 as needed. Due to the support provided by the hook clip 78, a relatively stable engagement of the endotracheal tube in the patient's mouth is maintained during the lateral sliding movement of the tube rest assembly 14.

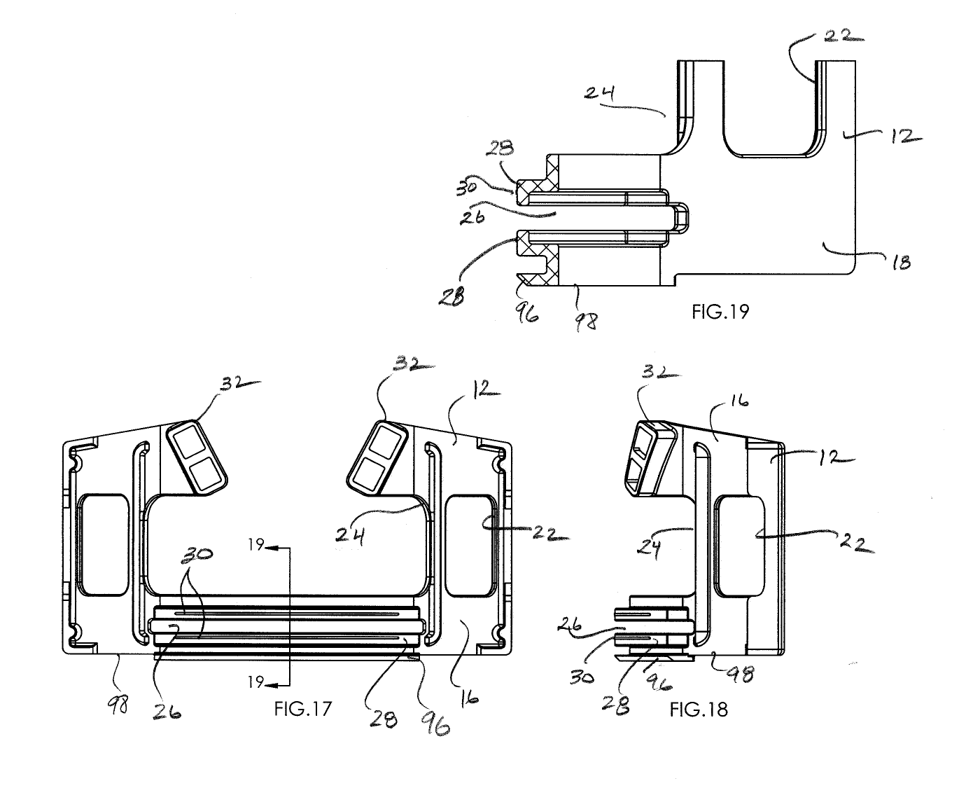

In addition, referring now to FIGS. 1 and 17-19, the face plate 12 is preferably provided with a shelf 96 projecting forwardly from a lower edge 98 of the face plate and past the front surface 16. The shelf 96 thus defines a groove for slidingly engaging a lower edge 100 of the tube rest assembly (FIGS. 8, 11, 12, 20) for still further preventing unwanted vertical movement and thus vertically stabilizing the tube rest assembly 14 relative to the face plate 12. It will be appreciated that the dimensions of the shelf 96 may vary to suit the application.

From the above description, it will be seen that the present respiratory facemask 10 provides smooth relative sliding motion of the tube rest assembly 14 relative to the face plate 12. The multiple points of slidable attachment, at the snap clip 46, the hook clip 78, the shelf 96 and the tabs 41 provide vertical sliding stability to the tube rest assembly 14, preventing unwanted forward or vertical movement. It is preferred that the components are manufactured to close tolerances, preferably approximately 0.002 inch to prevent undue play between components.

While particular embodiments of the present respiratory facemask with sliding endotracheal tube holder have been shown and described, it will be appreciated by those skilled in the art that changes and modifications may be made thereto without departing from the invention in its broader aspects and as set forth in the following claims.

What is claimed:

1. A respiratory therapy facemask configured to be positioned against a patient's face and adjacent the patient's mouth, comprising:
a face plate configured for attachment to the patient and defining an opening, said face plate including a laterally extending track defining a slot; and
a tube rest assembly configured for being slidably engaged in said slot and for securely accommodating an endotracheal tube, said tube rest assembly being configured for directly engaging said track for lateral movement in said opening.

2. The facemask of claim 1 wherein said track is provided with at least one rib for facilitating sliding action of said tube rest assembly relative to said face plate.

3. The facemask of claim 2 wherein said tube rest assembly includes a depending front panel having a rear surface that engages said track and slidably engages said at least one rib.

4. The facemask of claim 1 wherein said tube rest assembly includes a depending front panel configured for engaging a slide clip slidably associated with said track on a rear surface of said face plate.

5. The facemask of claim 4 wherein said slide clip has at least one prong matingly engaging an associated opening in said front panel.

6. The facemask of claim 4 wherein said face plate has a rear surface and said track includes at least one rib projecting from said rear surface for facilitating sliding action of said clip in said slot.

7. The facemask of claim 1 wherein said tube rest assembly includes a tube holder configured for receiving the endotracheal tube, said tube holder provided with at least one resilient pad upon which the tube is disposed.

8. The facemask of claim 7 wherein said at least one resilient pad is a relatively soft insert slidably engaged in a relatively hard plastic tube rest.

9. The facemask of claim 7 wherein said tube rest assembly is provided with a pair of locking straps configured to overlappingly engage each other for securing the tube in place.

10. The facemask of claim 1 wherein said tube rest assembly includes a tube holder provided with at least one rearwardly projecting rod, and at least one bite block pad associated with each said rod.

11. The facemask of claim 1 wherein said opening is dimensioned for accommodating lateral sliding action of said tube rest assembly, and said tube rest assembly is slidably secured to said face plate by at least one hook clip slidably engaging an edge of said face plate defining said opening, said at least one hook clip being constructed and arranged for overhanging said edge for preventing unwanted forward or vertical movement of said tube rest assembly relative to said face plate.

12. The facemask of claim 11 wherein said edge is located above said track.

13. The facemask of claim 1 wherein said face plate has a rear surface configured for contacting the patient's face, and being provided with at least one resilient pad.

14. The facemask of claim 1 further including at least one clip mount configured for accommodating a respective tube clip constructed and arranged for receiving and securing a second tube by a friction fit in a wedge-shaped portion.

15. A respiratory therapy facemask configured to be positioned against a patient's face and adjacent the patient's mouth, comprising:
a face plate configured for attachment to the patient and defining an opening, said face plate including a laterally extending track defining a slot;
a tube rest assembly configured for being slidably engaged in said slot and for securely accommodating an endotracheal tube; and
at least one clip provided for engaging said tube rest assembly from a rear side of said face plate for vertically stabilizing the tube rest as it laterally slides relative to said face plate.

16. The facemask of claim 15, further including a shelf projecting from a lower edge of said face plate past a front surface for vertically stabilizing said tube rest assembly.

17. The facemask of claim 15 further including a slide clip retaining said tube rest assembly to said face plate and sliding with said tube rest assembly in said slot, with said face plate sandwiched between said clip and said tube rest assembly; and
a hook clip engaging said tube rest assembly and having a hook clip slidably engaging a lower edge of said opening and preventing unwanted vertical and forward movement of said tube rest assembly relative to said face plate.

18. The facemask of claim 17, wherein at least one of said slide clip and said hook clip matingly engage said tube rest assembly respectively through at least one prong and at least one lug.

19. A respiratory therapy facemask configured to be positioned against a patient's face and adjacent the patient's mouth, comprising:
a face plate configured for attachment to the patient and defining an opening, said face plate including a laterally extending track defining a slot;
a tube rest assembly configured for being slidably engaged in said slot and for securely accommodating an endotracheal tube, at least one of said tube rest assembly and said face plate having ribs for facilitating the sliding engagement in said slot;
a slide clip retaining said tube rest assembly to said face plate and sliding with said tube rest assembly in said slot, with said face plate sandwiched between said clip and said tube rest assembly; and
a hook clip engaging said tube rest assembly and having a hook rib slidably engaging a lower edge of said opening and preventing unwanted vertical and forward movement of said tube rest assembly relative to said face plate.
